(19) United States
(12) Patent Application Publication
(10) Pub. No.: US 2004/0208844 A1
(43) Pub. Date: Oct. 21, 2004

(54) Products and drug delivery vehicles

(76) Inventor: Francis Ignatious, King of Prussia, PA (US)

Correspondence Address: SMITHKLINE BEECHAM CORPORATION, CORPORATE INTELLECTUAL PROPERTY-US, UW2220, P. O. BOX 1539, KING OF PRUSSIA, PA, 19406-0939, US

(21) Appl. No.: 10/485,023

(22) PCT Filed: Jul. 31, 2002
(86) PCT No.: PCT/US02/24423

Related U.S. Application Data
(60) Provisional application No. 60/309,363, filed on Aug. 1, 2001.

(51) Int. Cl. A61K 31/74

(52) U.S. Cl. 424/78.17

(57) ABSTRACT

Disclosed are products useful as, or in, drug delivry vehicles.

### FIELD OF INVENTION

The present invention relates to conjugates of (a) a polymeric component and (b) a non-biological, biomimetic antagonist to a receptor upregulated at a disease site. The conjugates are useful as, or in, drug delivery vehicles for drug delivery systems such as polymer-therapeutics and polymeric micelles, wherein the receptor antagonist imparts active targeting of the system to the disease site.

### BACKGROUND OF INVENTION

It is generally desirable to provide pharmaceutical actives in formulations targeted to the disease site in order to permit lower dosing, reduce side effects, and/or to improve patient compliance. This may be particularly true in the case of drugs that tend to have unpleasant side effects, especially when used at high doses, such as certain anti-cancer agents.

One approach to drug delivery are so-called “polymer-therapeutics”, which involve the association, e.g., by chemical conjugation, of a drug moiety to a polymer, e.g., in order to enhance the drug's circulation half-life and to reduce its toxicity. Examples of polymer-therapeutics include polyethylene glycol-conjugated proteins (aka pegylated proteins), including ONCOSPAR and ADAGEN.

Polymer-therapeutics may exhibit passive targeting, e.g., an enhanced permeability and retention (epr) effect, relating to passive accumulation at a tumor site through the leaky vasculature at the tumor site. One example of such polymer-therapeutics is SMANCS (low molecular weight styrene maleic anhydride copolymer conjugated to neocarzinostatin through the anhydride groups present in the polymer), an anti-tumor agent approved in Japan for liver cirrhosis. Other polymer-therapeutic systems have been investigated for passive targeting, e.g., polyhydroxypropylmethacrylamide(HPMA)-based drug conjugates, and polymeric micelles based on amphiphilic block copolymers derived from hydrophilic polyalkylene oxides (e.g., PEG), and hydrophobic polymers such as polypropylene glycol, polyesters, polycarbonates, derivatized poly(alpha-amino acid), poly(vinyl N-heterocycle) segments, and polynucleotide compositions.

Biorecognizable (targeting) ligands have also been investigated for site-specific delivery of pharmaceuticals. Targeting moieties have included, for example, proteins, monoclonal and polyclonal antibodies, carbohydrates, peptides, hormones, growth factors, vitamins, steroids, steroid analogs, cofactors, bioactive agents, and genetic material, including nucleosides, nucleotides and polynucleotides. Such targeting ligands have been used to direct polymer-drug conjugates, liposomes and polymeric micelles to specific cell subsets.

Certain receptors, including integrins such as the vitronectin (αᵥβ₃) receptor, are upregulated on the surface of growing endothelial cells. Additionally, the progression of a cancerous tumor involves processes characterized by neovascularization (angiogenesis). Inhibition of this angiogenesis will limit tumor progression and formation and progression of metastases. On this basis, anti-angiogenic agents have been proposed for the treatment of cancer. For example, a peptide-drug conjugate that binds to the αᵥβ₃ and αᵥβ₅ receptors has been shown to be a very potent anti-angiogenic compound, as blocking the αᵥβ₃ or αᵥβ₅ receptors results in the death of proliferating endothelial cells. Pasqualini, R. et al., Nature Biotechnology, Vol. 15, pp. 542-546 (1997).

Non-peptide receptor antagonists selective for one or more integrins, such as the vitronectin receptor (αᵥβ₃) and αᵥβ₅ receptor, have been described. See, e.g., Nicolau, K. C. et al., Design, Synthesis and Biological Evaluation of Nonpeptide Integrin antagonists, Bioorganic & Medicinal Chemistry 6 (1998) 1185-1208. Recent PCT publications disclose pharmaceutically active compounds which inhibit the vitronectin receptor and which are useful for the treatment of inflammation, cancer, cardiovascular disorders, such as atherosclerosis and restenosis, and/or diseases wherein bone resorption is a factor, such as osteoporosis, including: PCT applications WO 96/00730, published Jan. 11, 1996; WO 97/24119, published Jul. 10, 1992; WO 98/14192, published Apr. 9, 1998; WO98/30542, published Jul. 16, 1998; WO99/15508, published Apr. 1, 1999; WO99/05232, published Sep. 16, 1999; WO00/33838, published Jun. 15, 2000; WO97/01540, published Jan. 16, 1997; WO99/15170, published Apr. 1, 1999; WO99/15178, published Apr. 1, 1999; WO00/07544, published Feb. 17, 2000; WO96/00574, published Jan. 11, 1996; WO97/24122, published Jul. 10, 1997; WO97/24124, published Jul. 10, 1997; and WO99/05107, published Feb. 4, 1999. Inhibitors of the vitronectin receptor are also disclosed in WO 00/35887, published Jun. 22, 2000.

There is an ongoing need to develop means of targeted delivery of pharmaceutical actives. The present invention involves the discovery that the delivery of a pharmaceutical active in polymer-therapeutics, such as polymeric micelles, to a disease site can be improved by incorporating a non-biological, biomimetic ligand to a receptor upregulated at the disease site into the polymer-therapeutic. The receptor antagonist imparts active targeting of the polymer-therapeutic to the disease site. The non-biological, biomimetic ligand tends to have certain advantages relative to prior means of targeted delivery. E.g., such ligands tend to provide simpler manufacturing relative to polypeptide targeting ligands, less antigenic potential relative to antibody ligands, and/or a lesser impact on HLB vs proteins, such that micelles may be more readily formed.

### SUMMARY OF INVENTION

The present invention relates to polymer-receptor antagonist conjugates comprising (a) a pharmaceutically acceptable, polymeric component and (b) a nonbiological, biomimetic antagonist to a receptor upregulated at a disease site. In a preferred embodiment, the polymeric component of the conjugate is an amphiphilic copolymer and the conjugate forms micelles in aqueous media.

The invention also relates to polymer-therapeutics comprising such conjugates or polymeric micelles, and a pharmaceutical active.

The invention also relates to a method of treating or diagnosing a disease characterized by upregulation of a receptor, comprising administering to a patient in need thereof a safe and effective amount of such a polymer-therapeutic, wherein the antagonist has binding affinity to the upregulated receptor.

The present invention also relates to a novel method for preparing an amphiphilic biodegradable polymer having carboxylic groups at the hydrophilic terminus.

Other aspects of the present invention will become apparent to those skilled in the art upon reading and understanding the following detailed description.

### DETAILED DESCRIPTION

All documents cited or referred to herein, including issued patents, published and unpublished patent applications, and other publications are hereby incorporated herein by reference as though fully set forth.

Conjugates of the present invention comprise (a) a pharmaceutically acceptable, polymeric component and (b) a nonbiological, biomimetic antagonist to a receptor upregulated at a disease site. The polymeric component may be a homopolymer or copolymer (including block, graft or random copolymers), natural or synthetic, and may be hydrophilic, hydrophobic, or comprise a combination of hydrophilic and hydrophobic segments (i.e., amphiphilic copolymers). Suitable polymeric components are capable of chemical conjugation with the receptor antagonist, preferably through covalent bonding. The polymeric component is pharmaceutically acceptable, in that it is not deleterious to the recipient thereof.

A variety of hydrophilic polymers and hydrophobic polymers are known in the art and are useful for the polymeric components and segments herein.

Examples of suitable hydrophilic polymers include:

polyalkyl ethers and alkoxy—capped analogs thereof (e.g., polyoxyethylene glycol, polyoxyethylene/propylene glycol, and methoxy or ethoxy—capped analogs thereof, especially polyoxyethylene glycol);

polyvinylpyrrolidones;

polyvinylalkyl ethers;

polyoxazolines, polyalkyl oxazolines and polyhydroxyalkyl oxazolines;

polyacrylamides, polyalkyl acrylamides, and polyhydroxyalkyl acrylamides (e.g., polyhydroxypropylmethacrylamide and derivatives thereof);

polyhydroxyalkyl acrylates;

polysialic acids and analogs thereof;

hydrophilic peptide sequences;

polysaccharides and their derivatives, including

dextran and dextran derivatives, e.g., carboxymethyldextran, dextran sulfates, aminodextran;

cellulose and its derivatives, e.g., carboxymethyl cellulose, hydroxyalkyl celluloses;

chitin and its derivatives, e.g., chitosan, succinyl chitosan, carboxymethylchitin, carboxymethylchitosan;

hyaluronic acid and its derivatives;

starches;

alginates;

chondroitin sulfate;

albumin;

pullulan and carboxymethyl pullulan;

polyaminoacids and derivatives thereof, e.g., polyglutamic acids, polylysines, polyaspartic acids, polyaspartamides;

maleic anhydride copolymers such as:

styrene maleic anhydride copolymer,

divinylethyl ether maleic anhydride copolymer,

polyvinylalcohols;

copolymers thereof; and

derivatives of the foregoing.

The term “alkyl” and “alkoxy” includes C1-4, e.g., methyl, ethyl, propyl, dimethyl, and propylmethyl, and corresponding alkoxy groups.

Examples of suitable hydrophobic polymers include:

polyesters, e.g., polylactic acid, polymalic acid, polycaprolactone, polydioxanone,

polycarbonates,

polyanhydrides,

polyorthoesters;

hydrophobic derivatives of poly(alpha-amino acids) such as described for hydrophilic polymers;

polyalkyl ethers (e.g., polypropylene glycols);

copolymers thereof; and

derivatives of the foregoing.

In a preferred embodiment, the polymeric component comprises at least one hydrophilic segment. Without intending to be bound or limited by theory, drug delivery vehicles comprising such polymeric components tend to exhibit increased water solubility, increased circulation half-life, increased accumulation at the disease site, and/or reduced drug toxicity. Preferred hydrophilic polymeric components are water-soluble and non-antigenic.

In particularly preferred embodiments, the polymeric component is capable of forming polymeric micelles in aqueous medium. Polymeric micelles may be formed under appropriate conditions from block or graft, amphiphilic copolymers. Amphiphilic copolymers in aqueous medium undergo micellization by aggregation of the hydrophobic domains, in a process of self-assembly. The amphiphilic copolymer will preferably comprise:

(a) a hydrophilic polymer segment selected from the group consisting of polyethylene glycol (PEG), polyvinylpyrrolidone (PVP), polyacrylamide (PA), poly (hydroxypropyl acrylamide), polyvinylalcohol (PVA), polysaccharides, polyaminoacids, polyoxazolines, and copolymers and derivatives thereof; and

(b) a hydrophobic polymer segment selected from the group consisting of polyesters, polycarbonates, polyanhydrides, polyorthoesters, polypropylene glycol, hydrophobic derivatives of poly(alpha-amino acids), and copolymers and derivatives thereof.

Suitable derivatives of polymeric components include synthetic modifications according to well-known techniques wherein one or more functional groups present on the polymeric backbone are derivatized, the polymeric backbone structure being generally retained.

Suitable polymeric components are those capable of chemical conjugation with the receptor antagonist, preferably through covalent bonding. If necessary, the polymeric component will be derivatized using standard synthetic chemistry techniques to provide functionality for chemical conjugation with the receptor antagonist, and optionally with a pharmaceutical active of interest. Preferred functionality of the polymeric component includes functional groups such as COOH, CHO, NCO, NH2, OH and SH. For polymeric micelles, preferred amphiphilic polymers are those having reactive functional groups at the hydrophilic terminus. This configuration enables chemical conjugation of the receptor antagonist to the hydrophilic terminus, such that the antagonist will be present at the extremities of the outer hydrophilic shell of the polymeric micelle, thereby better directing the polymeric micelle to the disease site where receptors are present.

Methods of preparing functionalized polymers are well known in the art, for example, as described in Kataoka et al., Makromol. chem., 1989, 190, 2041; U.S. Pat. No. 5,929,177 and U.S. Pat. No. 5,925,720.

The present invention also provides a novel method of preparing amphiphilic biodegradable polymers having carboxylic groups at the hydrophilic terminus, by a single step method, as shown in Scheme 1.

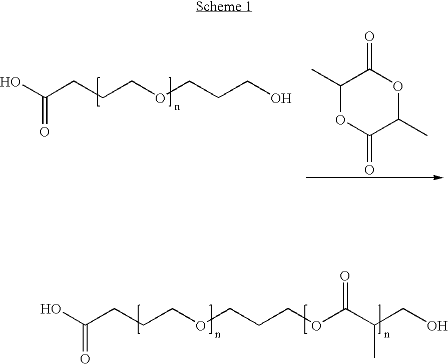

This one step synthesis comprises reacting a hydrophilic, alpha hydroxy omega carboxylic polyalkyleneglycol (preferably C2-4 alkylene, especially polyethyleneglycol), with a hydrophobic cyclic monomer such that ring opening polymerization of the monomer is initiated by the polyalkylene glycol hydroxy terminus. Hydrophobic cyclic monomer may be selected from propylene oxide, lactones (e.g., lactides, caprolactone, dioxanone, and their synthetic derivatives), cyclic carbonates (e.g., trimethylene carbonate and its derivatives), and combinations thereof.

Suitable alpha hydroxy omega carboxylic polyethyleneglycols are commercially available from Shearwater Polymers Inc., of Huntsville, Ala. (USA). Synthesis and purification of alpha hydroxy omega carboxylic polyethylene glycols is also described in U.S. Pat. No. 5,672,662, Harris et al. and in Journal of Bioactive and Compatible Polymers, 1990, 5, 227-231, Zalispky et al. Hydrophobic cyclic monomers are commercially available from a number of sources, e.g., lactides from Purac America (IL), caprolactone from Aldrich Chemical Co. (MN), and trimethylene carbonate from Boehringer Ingelheim (VA).

Ring opening polymerization techniques such as are known in the art may be employed to prepare the functionalized polymer. The ring opening polymerization may be carried out either in solution or melt, preferably in the melt. Catalysts, such as are known in the art, are preferably employed. Transition metal catalysts, e.g., stannous octoate, stannous chloride, zinc acetate, zinc, SnO, SnO₂, Sb₂O₃, PbO, and FeCl₃, are preferred, with stannous octoate more preferred. Other examples of suitable catalysts include GeO₂ and NaH. The polymerization reaction temperature will typically be from about 100 to about 200° C. As will be understood by those skilled in the art, the resulting polymer molecular weight will be determined by the molar ratio of the hydrophobic monomer to the hydroxy group present on the alpha hydroxy omega carboxylic polyalkylene glycol. The polymer molecular weight will typically be about 40,000 or less, although higher molecular weights may be used. This novel method desirably avoids polymer degradation, which might otherwise result when using a multiple step process involving protection and deprotection steps.

Receptor antagonists used in the present invention are small organic molecules that can bind a receptor upregulated at a disease site. The antagonists are non-biological, being synthetic material not isolated or derived from a biological source. Thus the present invention excludes peptides, antibodies, antibody fragments, vitamins and sugars, which are isolated or derived from biological sources. The antagonists are biomimetic, in that they bind a receptor. Preferred receptor antagonists have a high degree of selectivity and a high binding affinity to a receptor of interest.

Suitable non-biological, biomimetic antagonists for use in the present invention include those that bind to a receptor that is upregulated in the vascular endothelium of inflammation, infection or tumor sites. Examples of receptors that are upregulated in the vascular endothelium of inflammation, infection or tumor sites are integrin receptors, such as αVβ3, αVβ5 and α5β1, Prostate Specific Membrane Antigen (PSMA) receptor, Herceptin, Tie1 receptor, Tie2 receptor, ICAM1, Folate receptor, basic Fibroblast Growth Factor (bFGF) receptor, Epidermal Growth Factor (EGF) receptor, Vascular Endothelial Growth Factor (VEGF), Platelet Derived Growth Factor (PDGF) receptor, Laminin receptor, Endoglin, Vascular Cell Adhesion Molecule VCAM-1, E-Selectin, and P-Selectin.

Suitable non-biological, biomimetic antagonists include:

Analogs of YIGSR-NH2 (peptidomimetic inhibitors of the laminin receptor, such as described in Zhao M., Kleinman H K., and Mokotoff M., Synthesis and Activity of Partial Retro-Inverso Analogs of the Antimetastatic Laminin-Derived Peptide, YIGSR-NH2. International Journal of Peptide & Protein Research. 49(3):240-253, 1997 Mar.)

PD 156707 and derivatives thereof (such as described in Harland S P., Kuc R E., Pickard J D., Davenport A P. Expression of Endothelin (A) Receptors in Human Gliomas and Meningiomas, with High Affinity for the Selective Antagonist PD156707. Neurosurgery. 43(4):890-898, 1998 Oct.)

Integrin receptor antagonists, including antagonists to the receptors αVβ3 (vitronectin receptor), αVβ5 and α5β1.

Integrin receptor antagonists are preferred, antagonists to the receptors αVβ3, αVβ5 and α5β1, and especially αVβ3 being more preferred. Suitable integrin receptor antagonists include RGD mimetics.

Suitable receptor antagonists are those capable of chemical conjugation with the polymeric component, preferably through covalent bonding. If necessary, the receptor antagonist will be derivatized using conventional synthetic chemistry techniques to provide functionality for chemical conjugation with the polymeric component. Preferred functional groups are primary aliphatic (e.g., C3-C18) amines, carboxylic acids, sulfhydryls, or hydroxyls, more preferably amines or carboxylic acids. As will be understood by those skilled in the art, such derivatization will be designed so as to substantially retain the biomimetic character of the parent compound.

For example, RGD mimetics suitable for use in the present invention may be selected from the integrin receptor antagonists described in Nicolau, K. C. et al., Design, Synthesis and Biological Evaluation of Nonpeptide Integrin Antagonists, Bioorganic & Medicinal Chemistry 6 (1998)1185-1208, and in PCT applications WO 96/00730, published Jan. 11, 1996; WO 97/24119, published Jul. 10, 1992; WO 98/14192, published Apr. 9, 1998; WO98/30542, published Jul. 16, 1998; WO99/15508, published Apr. 1, 1999; WO99/05232, published Sep. 16, 1999; WO00/33838, published Jun. 15, 2000; WO97/01540, published Jan. 16, 1997; WO99/15170, published Apr. 1, 1999; WO99/15178, published Apr. 1, 1999; WO00/07544, published Feb. 17, 2000; WO96/00574, published Jan. 11, 1996; WO97/24122, published Jul. 10, 1997; WO97/24124, published Jul. 10, 1997; WO99/05107, published Feb. 4, 1999; PCT application No. PCT/US00/24514, filed Sep. 7, 2000; WO 00/35887, published Jun. 22, 2000; U.S. Pat. No. 5,929,120; and W. H. Miller et al., Identification and in vivo Efficacy of Small-Molecule Antagonists of Integrin αVβ3 (the Vitronectin Receptor), Drug Discovery Today, Vol. 5, Issue 9, Sep. 1, 2000, pp 397-408.

Examples of vitronectin receptor antagonists (“VRAs”) include compounds represented by the following structures:

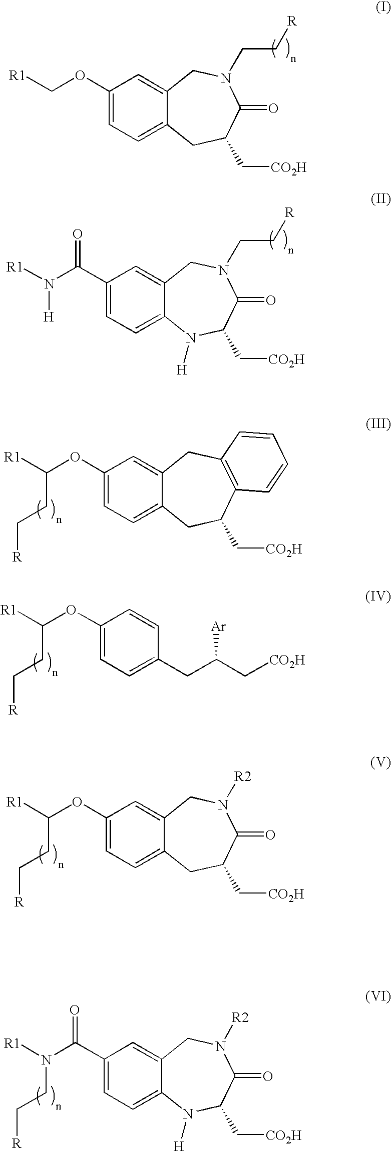

wherein the above structures (I)-(VI):

R1 selected from NH₂, COOH, and SH

R1 is selected from:

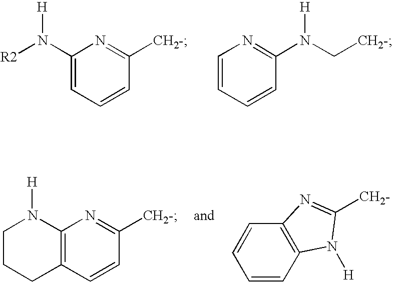

R2 is H or 1-4 C alkyl, especially H or CH3, and

n integer from 0-20, especially 0-5, e.g., 1-5.

Although many antagonists are contemplated herein, the subject invention is particularly disclosed using a vitronectin receptor antagonist having the structure:

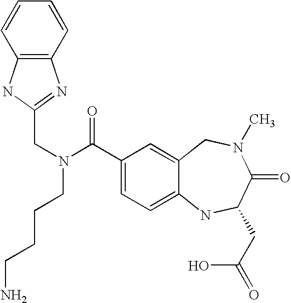

In another embodiment, the antagonist is the amino derivative of the structure:

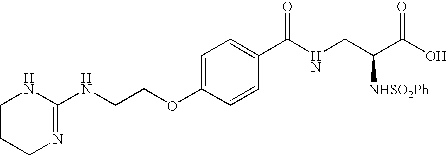

This compound and its synthesis is described in U.S. Pat. No. 5,929,120. The amino derivative can be readily prepared by one skilled in the art by substituting the phenyl sulfonyl with hydrogen, using standard synthetic chemistry techniques.

While such particular embodiments have been disclosed, it is to be understood that the present invention encompasses all antagonists to receptors upregulated at a disease site.

Conjugation of the polymeric component and receptor antagonist is preferably achieved by covalent bonding between functional groups on the polymeric component and functional groups on the receptor antagonist, e.g., to form non-biologically labile ester, amide or sulfonamide groups. In a preferred embodiment, the receptor antagonist is chemically conjugated to the hydrophilic terminus of an amphiphilic polymer. Methods suitable for achieving conjugation are known in the art, e.g., Zalipsky et al, Advanced drug delivery Reviews, 1995, 16, 157-182; and Eur. Polym. J. 19(12), 1177-1183, 1983. For example, chemical conjugation of the primary amino group of a receptor antagonist to the carboxylic group of an amphiphilic polymer can be performed by following the reaction Scheme 2. The carboxylic groups on the amphiphilic polymer are preactivated, e.g., by using N-hydroxysuccinimide in the presence of dicyclohexylcarbodiimide, and reacted with the primary amino group on the antagonist to form an amide bond. The synthesis is preferably carried out in organic medium under anhydrous conditions in the presence of a catalyst like dimethylaminopyridine or triethylamine.

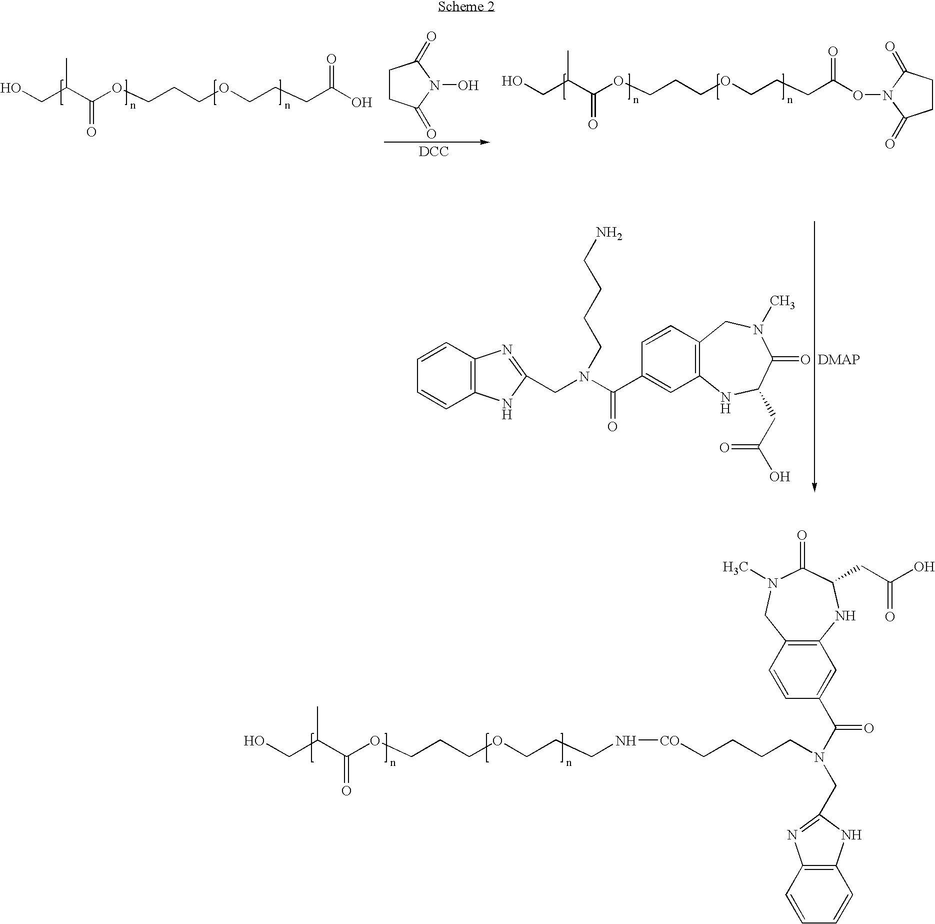

The polymer-receptor antagonist conjugates of the present invention are useful as, or in, drug delivery vehicles. In one embodiment, the conjugate is further chemically conjugated with a pharmaceutical active to form a polymer-therapeutic drug delivery system. In another embodiment, a polymer-receptor antagonist conjugate is used to prepare polymeric micelles that can be loaded with pharmaceutical active to form a drug delivery system.

Pharmaceutical actives include therapeutic agents and diagnostic agents. Therapeutic pharmaceutical actives may be selected, for example, from natural or synthetic compounds having the following activities: anti-angiogenic, anti-arthritic, anti-arrhythmic, anti-bacterial, anti-cholinergic, anti-coagulant, anti-diuretic, anti-epilectic, anti-fungal, anti-inflammatory, anti-metabolic, anti-migraine, anti-neoplastic, anti-parasitic, anti-pyretic, anti-seizure, anti-sera, anti-spasmodic, analgesic, anesthetic, beta-blocking, biological response modifying, bone metabolism regulating, cardiovascular, diuretic, enzymatic, fertility enhancing, growth-promoting, hemostatic, hormonal, hormonal suppressing, hypercalcemic alleviating, hypocalcemic alleviating, hypoglycemic alleviating, hyperglycemic alleviating, immunosuppressive, immunoenhancing, muscle relaxing, neurotransmitting, parasympathomimetic, sympathominetric plasma extending, plasma expanding, psychotropic, thrombolytic and vasodilating. The present invention may be especially useful for delivering cytotoxic therapeutic agents.

Examples of therapeutic agents that can be delivered include topoisomerase I inhibitors, topoisomerase I/II inhibitors, anthracyclines, vinca alkaloids, platinum compounds, antimicrobial agents, quinazoline antifolates thymidylate synthase inhibitors, growth factor receptor inhibitors, methionine aminopeptidase-2 inhibitors, angiogenesis inhibitors, coagulants, cell surface lytic agents, therapeutic genes, plasmids comprising therapeutic genes, Cox II inhibitors, RNA-polymerase inhibitors, cyclooxygenase inhibitors, steroids, and NSAIDs (nonsteroidal anti-inflammatory agents).

Specific examples of therapeutic agents include:

Topoisomerase I-inhibiting camptothecins and their analogs or derivatives, such as SN-38 ((+)-(4S)-4,11-diethyl-4,9-dihydroxy-1H-pyrano[3′,4′:6,7]-indolizine[1,2-b]quinoline-3,14(4H,12H)-dione); 9-aminocamptothecin; topotecan (hycamtin; 9-dimethyl-aminomethyl-10-hydroxycamptothecin); irinotecan (CPT-11; 7-ethyl-10-[4-(1-piperidino)-1-piperidino]-carbonyloxy-camptothecin), which is hydrolyzed in vivo to SN-38); 7-ethylcamptothecin and its derivatives (Sawada, S. et al., Chem. Pharm. Bull., 41(2):310-313 (1993)); 7-chloromethyl-10,11-methylene-dioxy-camptothecin; and others (SN-22, Kunimoto, T. et al., J. Pharmacobiodyn., 10(3): 148-151 (1987); N-formylamino-12,13,dihydro-1,11-dihydroxy-13-(beta-D-glucopyransyl)-5H-indolo[2,3-a]pyrrolo[3,4-c]carbazole-5,7(6H)-dione (NB-506, Kanzawa, G. et al., Cancer Res., 55(13):2806-2813 (1995); DX-8951f and lurtotecan (GG-211 or 7-(4-methylpiperazino-methylene)-10,11-ethylenedioxy-20(S)-camptothecin) (Rothenberg, M. L., Ann. Oncol., 8(9):837-855 (1997)); 7-(2-(N-isopropylamino)ethyl)-(20S)-camptothecin (CKD602, Chong Kun Dang Corporation, Seoul Korea); BN 80245, a beta hydroxylactone derivative of camptothecin (Bigg, C. H. et al., Biorganic &Medicinal Chemistry Letters, 7(17): 2235-2238, 1997);

Topoisomerase I/II-inhibiting compounds such as 6-[[2-dimethylamino)-ethyl]amino]-3-hydroxy-7H-indeno[2,1-c]quinolin-7-one dihydrochloride, (TAS-103, Utsugi, T., et al., Jpn. J. Cancer Res., 88(10):992-1002 (1997)); 3-methoxy-11H-pyrido[3′,4′-4,5]pyrrolo[3,2-c]quinoline-1,4-dione (Azal Q D, Riou, J. F., et al., Mol. Pharmacol., 40(5):699-706 (1991));

Anthracyclines such as doxorubicin, daunorubicin, epirubicin, pirarubicin, and idarubicin;

Vinca alkaloids such as vinblastine, vincristine, vinleurosine, vinrodisine, vinorelbine, and vindesine;

Platinum compounds such as cisplatin, carboplatin, ormaplatin, oxaliplatin, zeniplatin, enloplatin, lobaplatin, spiroplatin, ((−)-(R)-2-aminomethylpyrrolidine (1,1-cyclobutane dicarboxylato)platinum), (SP-4-3(R)-1,1-cyclobutane-dicarboxylato(2-)-(2-methyl-1,4-butanediamine-N,N)platinum), nedaplatin, and (bis-acetato-ammine-dichloro-cyclohexylamine-platinum(IV));

Anti-microbial agents such as gentamicin and nystatin;

Quinazoline antifolates thymidylate synthase inhibitors such as described by Hennequin et al. Quinazoline Antifolates Thymidylate Synthase Inhibitors: Lipophilic Analogues with Modification to the C2-Methyl Substituent (1996) J. Med. Chem. 39, 695-704;

Growth factor receptor inhibitors such as described by: Sun L. et al., Identification of Substituted 3-[(4,5,6,7-Tetrahydro-1H-indol-2-yl)methylene]-1,3-dihydroindol-2-ones as Growth Factor Receptor Inhibitors for VEGF-R2 (Flk-1/KDR), FGF-R1, and PDGF-Rbeta Tyrosine Kinases (2000) J. Med. Chem. 43:2655-2663; and Bridges A. J. et al. Tyrosine Kinase Inhibitors. 8. An Unusually Steep Structure-Activity Relationship for Analogues of 4-(3-Bromoanilino)-6,7-dimethoxyquinazoline (PD 153035), a Potent Inhibitor of the Epidermal Growth Factor Receptor (1996) J. Med. Chem. 39:267-276,

Inhibitors of angiogenesis, such as angiostatin, endostatin, echistatin, thrombospondin, plasmids containing genes which express anti-angiogenic proteins, and methionine aminopeptidase-2 inhibitors such as fumagillin, TNP-140 and derivatives thereof;

and other therapeutic compounds such as 5-fluorouracil (5-FU), mitoxanthrone, cyclophosphamide, mitomycin, streptozocin, mechlorethamine hydrochloride, melphalan, cyclophosphamide, triethylenethiophosphoramide, carmustine, lomustine, semustine, hydroxyurea, thioguanine, decarbazine, procarbazine, mitoxantrone, steroids, cytosine arabinoside, methotrexate, aminopterin, motomycin C, demecolcine, etopside, mithramycin, Russell's Viper Venom, activated Factor IX, activated Factor X, thrombin, phospholipase C, cobra venom factor [CVF], and cyclophosphamide.

In particular embodiments of the present invention, the therapeutic agent is selected from: a) antineoplastic agents, e.g., camptothecin or analogs thereof, such as topotecan doxorubicin, daunorubicin, vincristine, mitoxantrone, carboplatin and RNA-polymerase inhibitors, especially camptothecin or analogs thereof, and more especially topotecan; b) anti-inflammatory agents, e.g., cyclooxygenase inhibitors, steroids, and NSAIDs; c) anti-angiogenesis agents, e.g., fumagillin, tnp-140, cyclooxygenase inhibitors, angiostatin, endostatin, and echistatin; d) anti-infectives; and e) combinations thereof.

Examples of diagnostic agents include contrast agents for imaging including paramagnetic, radioactive or fluorogenic ions. Specific examples of such diagnostic agents include those disclosed in U.S. Pat. No. 5,855,866 issued to Thorpe et al. on Jan. 5, 1999.

Chemical conjugation of a polymer-receptor antagonist conjugate and a pharmaceutical active to form a polymer-therapeutic is preferably achieved by covalent bonding between at least one functional group on the polymeric component of the conjugate and at least one functional group on the pharmaceutical active, typically to form an ester, amide, urethane, hydrazone, thioether, carbonate, azo, imine (Schiff's base), carbon-carbon or disulfide bond. The linkage between the polymer and pharmaceutical may be designed according to known principles to be biologically labile if necessary, such that the pharmaceutical is chemically free to exhibit the desired pharmaceutical effect. For example, the linkage may be designed so as to undergo cleavage under acidic or enzymatic conditions. Suitable methods and reaction conditions for chemical coupling of a pharmaceutical and a polymer are summarized in reviews by R. Duncan et al., Encyclopedia of Controlled Drug Delivery, Vol. 2 p. 786 (E. Mathiowitz, editor); and by Kopecek et al., Advances in Polymer Science, 1995 (112), 55-123. If necessary, pharmaceutical actives can be derivatized by known synthetic chemistry techniques to provide the desired functionality, provided that the active remains pharmaceutically effective.

Polymeric micelles can be prepared from a polymer-receptor antagonist conjugate comprising an amphiphilic copolymer as the polymer component. Methods of making polymeric micelles are well known in the art, e.g., as described in M. C. Jones and J. C. Leroux, European Journal of Pharmaceutics and Biopharmaceutics, 48 (1999), 101-111. In general, polymeric micelles are formed by dissolving a lyophilized powder of the amphiphilic polymer at a concentration greater than its critical micelle concentration (cmc), the micelles being formed by a spontaneous self-assembly process. Such micelles will have a hydrophobic core and hydrophilic outer domain. The inventive polymer-receptor antagonist conjugates comprising an amphiphilic copolymer also spontaneously form polymeric nicelles by dissolving a lyophilized powder of the conjugate at a concentration greater than its cmc. The micelles have a hydrophobic core and a hydrophilic outer domain. In preferred embodiments, where the receptor antagonist is conjugated to the hydrophilic terminus of the amphiphilic polymeric copolymer, the antagonist will be situated in the hydrophilic outer domain.

In addition to the polymer-receptor antagonist conjugate, polymeric micelles of the present invention may optionally comprise other amphiphilic polymeric components capable of forming polymeric micelles, such as are known in the art. Nonlimiting examples of such other polymeric micellar systems include:

block copolymers of polyoxyethylene with hydrophobic polyoxyalkylene;

copolymers of polyoxyethylene with hydrophobic poly(alpha-aminoacids) or derivatives thereof; and

biodegradable amphiphilic copolymers, comprising a hydrophobic biodegradable polymer such as poly(lactic acid)(PLA), poly(glycolic acid)(PGA), polycaprolactone(PC), polyhydroxybutyric acid or polycarbonate coupled to a hydrophilic pharmaceutically acceptable polymer such as PEG, polyvinylpyrrolidone, polyvinylalcohol, dextran, alginic acid, gelatin, pluronic etc.

A suitable pharmaceutical active is associated with the polymeric micelles. For example, a hydrophobic active can be associated with the hydrophobic inner core of the polymeric micelles in aqueous medium, by specific interactions such as hydrophobic association or chemical conjugation through a labile bond, depending on the nature of the pharmaceutical active and polymeric micelle. Hydrophobic actives include otherwise hydrophilic actives that are rendered hydrophobic, e.g., by conjugation with hydrophobic polymers by known methods. Physical entrapment of a hydrophobic pharmaceutical active in the hydrophobic inner core of polymeric micelles via hydrophobic association may be achieved by dialysis or emulsification techniques such as described in European Journal of Pharmaceutics and Biopharmaceutics, 48:, 101-111, 1999, J. C. Leroux et al., and WO 97/10849, Kim et al. Generally, the hydrophobic pharmaceutical active and polymer-receptor antagonist conjugate are dissolved in a suitable organic medium to solubilize the active and conjugate, and the solution is then dialyzed against water and lyophilized. The lyophilized powder may then be used to form polymeric micelles comprising the hydrophobic pharmaceutical and the receptor antagonist.

Pharmaceutical actives may be chemically conjugated to the amphiphilic polymer where each reactant has one or more appropriate functional groups. Chemical conjugation of pharmaceuticals to polymeric micellar carriers may be accomplished, e.g., by methods described in Journal of Controlled Release, 50, (1-3), 79-92 1998, Kataoka et al, and Colloids and Surfaces B: Biointerfaces, 16, (14): 217-2261999, Kwon et al.

In order to use the drug delivery systems of the present invention, they will normally be formulated into a pharmaceutical composition, in accordance with standard pharmaceutical practice. This invention therefore also relates to a pharmaceutical composition, comprising (a) an effective, non-toxic amount of a drug delivery system herein described and (b) a pharmaceutically acceptable carrier or diluent.

The pharmaceutical compositions may conveniently be administered by any of the routes conventionally used for drug administration, for instance, parenterally, orally, topically, by inhalation (e.g., inter-tracheally), subcutaneously, intramuscularly, inter-lesionally (e.g., to tumors), inter-nasally, intra-ocularly, by direct injection into organs, and intra-venously. Parenteral, particularly intravenous, administration is preferred.

The pharmaceutical composition may be in conventional dosage forms prepared by combining the drug delivery system with standard pharmaceutical carriers according to conventional procedures. The pharmaceutical composition may also comprise one or more other pharmaceutical active compounds, in conventional dosages. Preparation of the dosage form may involve mixing, granulating and compressing or dissolving the ingredients as appropriate to the desired preparation.

It will be appreciated that the form and character of the pharmaceutically acceptable carrier or diluent is dictated by the amount of drug delivery system and other active agents with which it is to be combined, the route of administration and other well-known variables. The carrier(s) or diluent(s) must be “acceptable” in the sense of being compatible with the other ingredients of the formulation and not deleterious to the recipient thereof. The drug delivery systems of the present invention will typically be provided in suspension form in a liquid carrier such as aqueous saline or buffer.

In general, the pharmaceutical dosage form will comprise the drug delivery system in an amount sufficient to deliver it in the desired dosage amount and regimen.

The pharmaceutical composition is administered in an amount sufficient to deliver the pharmaceutical active in the desired dosage according to the desired regimen, to ameliorate or prevent the disease state which is being treated, or to image the disease site being diagnosed or monitored.

It will be recognized by one of skill in the art that the optimal quantity and spacing of individual dosages of the pharmaceutical composition will be determined by the nature and extent of the condition being treated, diagnosed or monitored, the form, route and site of administration, and the particular patient being treated, and that such optimums can be determined by conventional techniques. It will also be appreciated by one of skill in the art that the optimal course of treatment, i.e., the number of doses given per day for a defined number of days, can be ascertained by those skilled in the art using conventional course of treatment determination tests.

Once administered, the drug delivery system associates with the targeted tissue, or is carried by the circulatory system to the targeted tissue, where it associates with the tissue. At the targeted tissue site, the receptor antagonist may itself exhibit clinical efficacy in treating a disease presenting the targeted receptor. In addition or alternatively, the pharmaceutical active associated with the drug delivery system is released or diffuses to the targeted tissue where it performs its intended function.

As will be appreciated by those skilled in the art, the design and selection of a particular drug delivery system is based on the expression of the conjugate's cognate receptor on a patient's diseased cells, and the activity of a particular pharmaceutical active in treating or diagnosing the disease. The expression of the cognate receptor and activity of the pharmaceutical active can be determined by known methods or may be based on historical information for the disease and active. Selection of a particular pharmaceutical active will be made depending on the disease being treated or diagnosed, including the nature of the disease site and the activity of the active toward that site, which may be based, for example, on chemosensitivity testing according to methods known in the art, or on historical information and accepted clinical practice.

For example, drug delivery systems comprising a receptor antagonist to receptors upregulated in the vascular endothelium of disease sites, such as inflammation, infection or tumor sites (e.g., the vitronectin receptor), are useful for treating diseases characterized by neovascularization (angiogenesis). Such diseases include osteo and rheumatoid arthritis, diabetic retinopathy, hemangiomas, psoriasis, restenosis and cancerous tumors (solid primary tumors as well as metastatic disease). The receptor antagonist binds the vitronectin receptor present at the disease site to target the pharmaceutical active to the disease site (the antagonist may also inhibit formation of vasculature). For treating or diagnosing such diseases, the drug delivery system will preferably comprise a therapeutic agent and/or diagnostic agent selected from the group consisting of anti-inflammatory agents, anti-neoplastic agents, anti-infectives, anti-angiogenic agents, and/or a diagnostic imaging agent. Selection of an active agent will be made based on the nature of the disease site (e.g., tumor, inflammation or infection) and the activity of the agent toward that site (e.g., anti-neoplastic, anti-inflammatory, anti-infective, respectively). Selection of a particular active may be based on chemosensitivity testing according to methods known in the art, or may be based on historical information and accepted clinical practice. For example, topotecan is known to be an active agent against ovarian cancer, and therefore is useful for treatment of ovarian cancer based on accepted clinical practice.

### EXAMPLES

The following examples illustrate the present invention. It should be noted that the present invention is not limited by these examples.

1) Preparation of the Vitronectin Receptor Antagonist (S)-7-[[N-(4-Aminobutyl)-N-(benzimidazol-2-ylmethyl)]amino]carbonyl-4-methyl-3-oxo-2,3,4,5-tetrahydro-1H-1,4-benzodiazepine-2-acetic acid (hereinafter “VRA 1”): General

Proton nuclear magnetic resonance (¹H NMR) spectra are recorded at either 300 or 400 MHz, and chemical shifts are reported in parts per million (δ) downfield from the internal standard tetramethylsilane (TMS). Mass spectra are obtained using electrospray (ES) ionization techniques. Elemental analyses are performed by Quantitative Technologies Inc., Whitehouse, N.J. All temperatures are reported in degrees Celsius. Analtech Silica Gel GF and E. Merck Silica Gel 60 F-254 thin layer plates are used for thin layer chromatography. Flash chromatography is carried out on E. Merck Kieselgel 60 (230-400 mesh) silica gel. Analytical and preparative HPLC is performed on Beckman chromatography systems. ODS refers to an octadecylsilyl derivatized silica gel chromatographic support. YMC ODS-AQ® is an ODS chromatographic support and is a registered trademark of YMC Co. Ltd., Kyoto, Japan. PRP-1® is a polymeric (styrene-divinylbenzene) chromatographic support, and is a registered trademark of Hamilton Co., Reno, Nev. Celite® is a filter aid composed of acid-washed diatomaceous silica, and is a registered trademark of Manville Corp., Denver, Colo.

The title compound is synthesized in accordance with the following scheme:

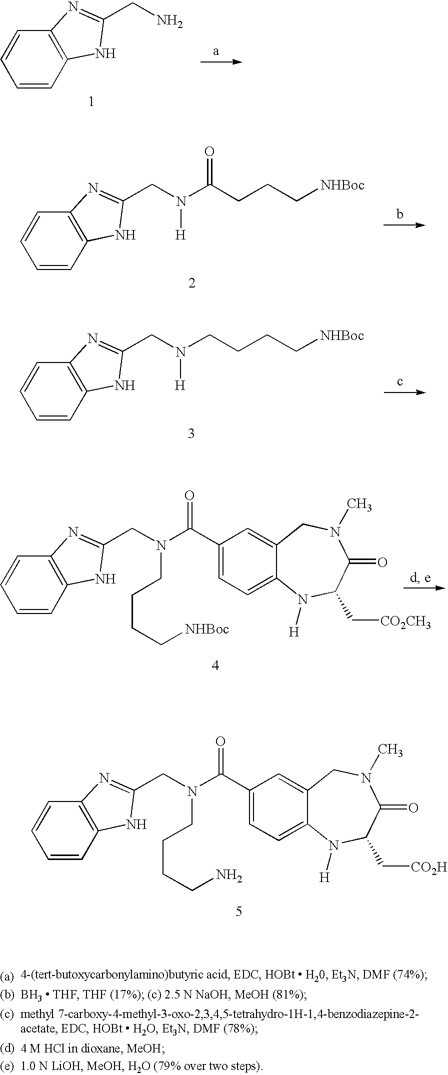

a) N-(Benzimidazol-2-ylmethyl)-4-(tert-butoxycarbonylamino)butyramide

4-(tert-Butoxycarbonylamino)butyric acid (5.0 g, 24.6 mmole), 2-aminomethylbenzimidazole dihydrochloride hydrate (6.5 g, 29.5 mmole), EDC (5.7 g, 29.5 mmole), HOBt.H₂O (3.99 g, 29.5 mmole), and Et₃N (17 mL, 123 mmole) are combined in DMF (120 mL) at RT. The reaction is stirred for 18 hr, then is concentrated to dryness. The residue is purified by flash chromatography on silica gel to afford the title compound (6.04 g, 74%): ¹H NMR (400 MHz, CDCl₃).7.40-7.80 (m, 2H), 7.29-7.38 (m, 1H), 7.20-7.27 (m, 2H), 4.77-4.90 (m, 1H), 4.69 (d, J=5.8 Hz, 2H), 3.11-3.22 (m, 2H), 2.20-2.39 (m, 2H), 1.77-1.88 (m, 2H), 1.44 (s, 9H).

b) N-(Benzimidazol-2-ylmethyl)-N-[4-(tert-butoxycarbonylamino)butyl]amine

Borane-tetrahydrofuran complex (1.0 M in THF, 55 mL, 55 mmole) is added slowly to a suspension of N-(benzimidazol-2-ylmethyl)-4-(tert-butoxycarbonylamino)butyramide (6.04 g, 18.2 mmole) in THF (90 mL) at RT. The resulting homogeneous solution is heated at reflux for 18 hr, then cooled to RT. A solution of 5% AcOH in EtOH is added, and the solution is stirred for 18 hr. The resulting solution is concentrated to dryness and the residue is taken up in saturated NaHCO₃. The mixture is extracted with CH₂Cl₂ (4×), and the combined organic layers are dried (MgSO₄) and concentrated. Flash chromatography on silica gel (10% MeOH/CH₂Cl₂) gives the title compound (985 mg, 17%) as a light tan gum: ¹H NMR (400 MHz, CDCl₃) δ 7.53-7.63 (m, 2H), 7.18-7.30 (m, 2H), 4.12 (s, 2H), 3.00-3.18 (m, 2H), 2.65-2.75 (m, 2H), 1.35-1.63 (m, 13H).

c) Methyl (S)-7-[[N-(benzimidazol-2-ylmethyl)-N-[4-(tert-butoxycarbonylamino)butyl]amino]carbonyl-4-methyl-3-oxo-2,3,4,5-tetrahydro-1H-1,4-benzodiazepine-2-acetate

Methyl 7-carboxy-4-methyl-3-oxo-2,3,4,5-tetrahydro-1H-1,4-benzodiazepine-2-acetate is synthesized by the method described in William H Miller, et al.: Enantiospecific Synthesis of SB 214857, a Potent, Orally Active, Nonpeptide Fibrinogen Receptor Antagonist Tetrahedron Letters (1995) 36(52): 9433-9436.

Methyl 7-carboxy-4-methyl-3-oxo-2,3,4,5-tetrahydro-1H-1,4-benzodiazepine-2-acetate (753 mg, 2.6 mmole), N-(benzimidazol-2-ylmethyl)-N-[4-(tert-butoxycarbonylamino)butyl]amine (985 mg, 3.1 mmole), EDC (594 mg, 3.1 mmole), HOBt.H₂O (419 mg, 3.1 mmole), and Et₃N (0.90 mL, 6.5 mmole) are combined in DMF (15 mL) at RT. The reaction is stirred for 18 hr, then is concentrated to dryness. The residue is purified by flash chromatography on silica gel (5% MeOH/CH₂Cl₂) to afford the title compound (1.2 g, 78%) as a light tan solid: ¹H NMR (400 MHz, CDCl₃) δ 10.55 (br s, 1H), 7.75 (d, J=8.5 Hz, 1H), 7.45 (d, J=8.5 Hz, 1H), 7.20-7.32 (m, 2H), 7.10-7.20 (m, 2H), 6.52 (d, J=8.1 Hz, 1H), 5.43 (d, J=16.5 Hz, 1H), 5.02-5.12 (m, 1H), 4.73-4.85 (m, 2H), 4.55-4.65 (m, 1H), 4.49 (d, J=4.7 Hz, 1H), 3.74 (s, 3H), 3.70 (d, J=16.5 Hz, 1H), 3.36-3.46 (m, 2H), 3.04 (s, 3H), 2.90-3.10 (m, 3H), 2.67 (dd, J=16.0, 6.4 Hz, 1H), 1.60-1.75 (m, 2H), 1.43 (s, 9H), 1.17-1.32 (m, 2H); MS (ES) m/e 593 (M+H)⁺.

d) (S)-7-[[N-(4-Aminobutyl)-N-(benzimidazol-2-ylmethyl)]amino]carbonyl-4-methyl-3-oxo-2,3,4,5-tetrahydro-1H-1,4-benzodiazepine-2-acetic Acid

4 M HCl in dioxane (30 mL, 120 mmole) is added to a solution of methyl (S)-7-[[N-(benzimidazol-2-ylmethyl)-N-[4-(tert-butoxycarbonylamino)butyl]amino]carbonyl-4-methyl-3-oxo-2,3,4,5-tetrahydro-1H-1,4-benzodiazepine-2-acetate (1.2 g, 2 mmole) in MeOH (10 mL) at RT. After 2 hr, the solution is concentrated to dryness to leave an off-white powder (1.24 g). This powder is dissolved in MeOH/H₂O (10 mL), and 1.0 N LiOH (10 mL, 10 mmole) is added. The reaction is stirred at RT for 18 hr, then concentrated to dryness. The residue is taken up in H₂O and the pH is adjusted to about 5 with 10% HCl. The precipitated solid is collected by suction filtration and washed with H₂O. Drying in high vacuum gives the title compound (760 mg, 79%) as a white solid: ¹H NMR (400 MHz, CDCl₃).7.48-7.68 (m, 2H), 7.05-7.35 (m, 4H), 6.57 (d, J=8.2 Hz, 1H), 5.51 (d, J=16.0 Hz, 1H), 5.12 (t, J=6.8 Hz, 1H), 4.70-5.00 (m, 2H, obscured by residual solvent signal), 3.62-3.90 (m, 1H), 3.40-3.62 (m, 2H), 2.95 (s, 3H), 2.69-3.00 (m, 3H), 2.45 (dd, J=15.6, 6.6 Hz, 1H), 1.60-1.80 (m, 2H), 1.30-1.60 (m, 2H); MS (ES) m/e 479 (M+H)⁺. Anal. Calcd for C₂₅H₃₀N₆O₄.2H₂O: C, 58.35; H, 6.63; N, 16.33. Found: C, 58.17; H, 6.63; N, 16.11.

Analogous vitronectin receptor antagonists having a functional aliphatic carboxylic acid group or aliphatic sulfhydryl group instead of the aliphatic amino group can be prepared in a similar manner, substituting the appropriate carboxylic acid in step (a) and utilizing the solvents 4M HCl in dioxane, CH₂Cl₂ in step (d).

2) Synthesis of Alpha Hydroxy Omega Carboxylic Terminated Amphiphilic Block Copolymers

Block copolymers A and B were synthesized starting from alpha hydroxy omega carboxylic terminated polyethylene glycol, available from Polymer Sources (Canada), Number Average Molecular Weight Mn=2100, Weight Average Molecular weight Mw=2450, Carboxylic Functionality by acidimetric titration 98%. 20 g of this polymer was dried by azeotropic distillation under toluene using a Dean-Stark Apparatus, and the residual toluene was removed under vacuum. Reaction was carried out in silanized glass test tubes. The components were weighed out into a test tube, in a dry box filled with dry nitrogen.

For Polymer A, 5 g of dried alpha hydroxy omega carboxylic PEG and 5 g of dl-Lactide (Purac) were used. For Polymer B, 4 g of dried alpha hydroxy omega carboxylic PEG and 6 g of dl-Lactide were used. The test tubes were sealed with rubber septums. 0.5 ml of 0.01M stannous octaoate in dry toluene was added to the test tube using a syringe. The test tubes were put under vacuum and then purged with dry nitrogen gas three times. The test tubes were immersed in an oil bath at 160° C. When the contents were melted the tubes were taken out, and the contents were thoroughly mixed using a vibratory mixer. Polymerization was continued for 6 h at 160° C. Upon completion of the polymerization the test tubes were cooled and the polymers were recovered.

Nine grams of each polymer was separately dissolved in 50 ml of acetone, the acetone solutions were separately added to 700 ml isopropanol, and the resulting cloudy solutions were centrifuged. The residues were collected, dissolved in 20 ml of water and lyophilized.

Polymer Molecular weight was determined by a Shimadzu GPC system (Shimadzu LC-10AD Pump, SIL-10AXL Autosampler, SPD-10A UV detector, Waters 2410 refractive Index detector, Viscotek T60A dual detector). Data acquisition and processing is performed by Viscotek Trisec GPC 3.0 software using universal calibration mode.

Number average molecular weight (Mn) was determined by acidimetric titration, assuming the presence of one carboxylic group per polymer chain. About 0.2 g of the polymer was weighed and dissolved in milliQ water, and the solution was titrated against 0.01N Sodium Hydroxide solution using phenolphthalein as the indicator. Mn=wt. of the sample (g)X 1000/Volume of NaOH X Normality of NaOH.

Critical Micelle Concentration (cmc) was determined by a Kruss K12 Tensiometer using the Wilhemy plate method. Data acquisition and processing was done using K122 software. A polymer solution of known concentration was automatically titrated into milliQ water, and surface tension values were automatically recorded and plotted against respective concentration to yield the cmc. Size of the polymeric micelles was determined by a Malvern 5000 Zeta Sizer at a polymer concentration in water above the cmc.

The polymers exhibited the following properties:

| | GPC analysis | | Acidimetry | cmc | Particle Size |
|---|---|---|---|---|---|
| Polymer | Mn | Mw | Mn | mg/L | nm |
| A | 3820 | 6340 | 3168 | 186 | 15 |
| B | 4650 | 7990 | 4935 | 38 | 10 |

3) Conjugation of Receptor Antagonist VRA 1 and Polymer B

a) Method A—Coupling in the presence of dicyclocarbodimide and dimethylaminopyridine

VRA 1 was first converted to the sodium salt before coupling with the polymer. 104 mg of VRA 1 was dissolved in a mixture of methanol and water, and 17 mg of NaHCO3 was added to the solution. The solution was stirred for 1 h and then lyophilized to give a white powder.

Polymer B (0.5 g) was dried by azeotropic distillation under toluene. The dried polymer was dissolved in dry DMSO in a 50 ml round bottom flask, under dry nitrogen. 0.05 g of the sodium salt of VRA 1 was added to the polymer solution to form a clear solution. 0.021 g of dicyclohexylcarbodimide (Aldrich) and 0.012 g dimethylaminopyridine (Aldrich) were added to the solution. The reaction mixture was stirred overnight (about 12 hours) at room temperature under dry nitrogen atmosphere. The reaction was then quenched by adding 5 ml of milliQ water. This solution was dialyzed against 2L milliQ water for two days with frequent replacement of water, using 2K molecular weight cut off dialysis membrane (Spectropure). After dialysis was completed the sample was lyophilized to get a white powder. GPC analysis of the sample using a UV detector at 280 nm shows the presence of VRA 1 conjugated to the polymer and the absence of any residual unreacted VRA 1. Absence of free VRA 1 in the conjugate was also confirmed by an HPLC method, using C18 column and 80/20 acetonitrile/0.05M citric acid buffer in an isocratic mode at flow rate of 1 ml/min.

b) Method B—Conjugation using Succinimydyl Ester of Polymer B

The reaction was carried out in a 50 ml round bottom flask under dry nitrogen atmosphere. Polymer B (0.5 g) was dried by azeotropic distillation under toluene. The dry polymer was dissolved in 5 ml of dry tetrahydrofuran (THF). 20.6 mg dicyclohexylcarbodimide and 11.5 mg N-hydroxysuccinimide were then added to the polymer solution. The reaction mixture was stirred for 24 h. At the end of the reaction the precipitate formed was filtered off and then THF was removed under vacuum. A solution of 50 mg of VRA 1 and 12 mg dimethylaminopyridine in 10 ml dry DMSO was then added to the flask and the reaction mixture was stirred for another 12 h. At the end of the reaction the solution was transferred to a dialysis bag (1K molecular weight cut off), first dialyzed against 2L of Bup MES (Pierce) solution having a pH of 4.7, and then dialyzed against milliQ water. The dialyzed solution was collected and lyophilized. Analysis of the lyophilized sample by GPC coupled to a UV detector shows the absence of any residual VRA 1. Absence of free VRA 1 in the conjugate was also confirmed by an HPLC method, using C18 column and 80/20 acetonitrile/0.05M citric acid buffer in an isocratic mode at flow rate of 1 ml/min.

The amount of VRA 1 in the conjugates was determined by both nitrogen analysis and a UV spectroscopic method. For the UV method a calibration curve was constructed by determining the UV absorbance at 28 mm for known concentrations of VRA 1 in a 1:1 ethanol/water mixture; the polymer conjugates were prepared in the same solvent medium. Critical Micelle Concentration (cmc) of the conjugates was determined by tensiometry as described above. The conjugates exhibited the following properties:

| Polymer | Mole % of VRA 1 | | cmc |
|---|---|---|---|
| conjugate | Nitrogen analysis | UV spectroscopy | (mg/L) |
| Method A | 26 | 18 | 23 |
| Method B | 75 | 93 | 14 |

4) In Vitro Binding Assays

In vitro binding affinity of conjugates, polymeric micelles and polymer therapeutics of the present invention may be determined by receptor binding assays such as are known in the art. Conjugates, polymeric micelles and polymer therapeutics of the present invention will have a Ki (the dissociation constant of the antagonist) according to a receptor binding assay in the nanomolar to micromolar range, preferably in the nanomolar range.

The following samples were prepared for an in vitro binding assay:

Solution #1: polymer-receptor antagonist conjugate according to Example 3b, dissolved in TBS at a concentration of 10 milliMole of VRA 1.

Solution #2: PEG-PLA copolymer according to Example 2B, dissolved in TBS at a concentration of 50 mg/ml;

Solution #3: VRA 1 dissolved in 1:1 TBS:DMSO at a concentration of 10 milliMole;

Solution #4: polymer-receptor antagonist conjugate according to Example 3b, dissolved in 1:1 TBS:DMSO at a concentration of 10 milliMole VRA 1; and

Solution #5: PEG-PLA copolymer according to Example 2B, dissolved in 1:1 TBS:DMSO at a concentration of 50 mg/ml.

Binding studies were carried out according to the method described by Wong et al., Studies on alphavbeta3/ligand interactions using a (³H)SK&F-107260 binding assay, Mol. Pharmacology, 1996, 50, 529-537. Human placenta or human platelet vitronectin receptor, αvβ3 (0.12 ug) was added to 96-well plates at 100 ul per well and incubated over night at 4° C. At the time of experiment, the wells were aspirated and incubated in 0.1 ml of Buffer A (50 mM Tris, 100 mM NaCl, 1 mM MgCl₂,1 mM MnCl₂, pH 7.4) containing 3% BSA for 1 hour at room temperature to block the nonspecific binding sites. The blocking solution was then removed, and various concentrations of the 5 sample solutions and 5 nM [³H]-SK&F-107260 were added to the wells. After one hour incubation at room temperature, the wells were aspirated completely and washed twice with 100 ul of ice-cold Buffer A. Bound [³H]-SK&F-107260 was solubilized and counted.

PEG-PLA dissolved in TBS or TBS/DMSO did not exhibit binding activity. Ki of VRA 1 is 1.7 nM, and that of VRA 1 conjugated PEG-PLA is 21 nM in TBS and 30 nM in TBS/DMSO, respectively.

5) Conjugation of VRA 1 with Other Polymeric Components

a) Polyglutamic Acid-VRA 1 Conjugate

Poly(1-glutamic acid) (PG) sodium salt was obtained from Sigma (St. Louis, Mo.). Lot-specific polydispersity (M,/Mn) was 1. 15 where MW is weight-average molecular weight. PG sodium salt (MW 34 K, Sigma, 0.35 g) is first converted to PG in its proton form. The pH of the aqueous PG sodium salt solution is adjusted to 2.0 using 0.2 M HCl. The precipitate is collected, dialyzed against distilled water, and lyophilized to yield PG.

To a solution of PG (75 mg, repeating unit FW 170, 0.44 mmol) in dry N,N-dimethylformamide (DMF) (1.5 mL) is added 11 mg: sodium salt of VRA 1, 15 mg dicyclohexylcarbodiimide (DCC (0.073 mmol) and trace amount of dimethylaminopyridine (DMAP). The reaction is carried out for 24 h. The resulting solution is dialyzed against (1K molecular weight cut off) 2L of Bup MES (Pierce) solution having a pH of 4.7, and then dialyzed against milliQ water. The dialyzate is lyophilized to obtain the polyglutamate-VRA 1 conjugate.

b) Dextran-VRA 1 Conjugate

(i) Production of a Carboxymethylated Dextran Sodium Salt

40 g of sodium hydroxide is added to and dissolved into 200 ml of purified water while cooling over ice. Into the resultant solution is dissolved 10 g of dextran, (Sigma, St. Louis, Mo., average molecular weight 15-20K), to thereby obtain a mixture. To the obtained mixture is added 50 g of monochloroacetic acid at room temperature to effect a reaction for 20 hours, to thereby obtain a reaction mixture. The pH value of the obtained reaction mixture is adjusted to 8 with acetic acid. The reaction mixture having a pH value of 8 is poured into 1.5 liters of methanol, to thereby generate a precipitate. The generated precipitate is collected and dissolved in 200 ml of purified water, to thereby obtain a solution. The obtained solution was dialyzed against purified water using a dialysis membrane (cut off molecular weight: 12,000 to 14,000, manufactured and sold by Spectrum Medical Ind., Inc., U.S.A.) at 4° C. for two days, to thereby obtain a dialyzate. The obtained dialyzate is subjected to filtration using a membrane filter (pore size: 0.22 μm), followed by lyophilization to thereby obtain compound carboxymethyl dextran. The degree of carboxymethylation of the obtained compound per sugar residue can be obtained by potentiometric titration.

1 g of carboxymethylated dextran sodium salt obtained in step 1 is dissolved in 10 ml of water and acidified with 0.1N HCl to bring the pH to 2. The resultant solution is dialysed against milliqQ water and the dialyzate is lyophilized to obtain carboxymethyl dextran.

(ii) Conjugation of VRA 1 to Carboxymethyl Dextran

100 mg of carboxymethyl dextran is dissolved in 1 ml water. 10 mg of sodium salt of VRA 1 dissolved in 1 ml of DMF is added to the aqueous solution of carboxymethyl dextran. To this solution is added 1-(3-dimethylaminopropyl)-3-ethyl-carbodiimide. The reaction mixture is left stirring for 24 h and the resulting solution is first dialyzed against (1K molecular weight cut off), against 2L of Bup MES (Pierce) solution having a pH of 4.7, and then dialyzed against milliQ water. The dialyzate is lyophilized to obtain the carboxymethyl dextran VRA 1 conjugate.

c) HPMA-VRA 1 Conjugate

Copolymer of N-(2-hydroxypropyl)methacrylamide and N-methacryloylglycine p-nitrophenylester (0.15 g), is prepared as described in Makromol. Chem., 178, 2159 (1977), containing 2.7×10³ equivalents of p-nitrophenyl ester, and reacted with VRA 1 (18 mg), in dry dimethylsulfoxide 5 ml) at room temperature for 18 hours, then with 1-amino-2-propanol for one hour at room temperature. The reaction mixture is treated with acetone (70 ml). The precipitate is collected, redissolved with anhydrous ethanol (5 ml) and reprecipitated with acetone (50 ml) to give the HPMA-receptor antagonist conjugate.

d) Chitosan-VRA 1 Conjugate

(i) Depolymerization of Chitosan

Chitosan (Protan, Inc., Portsmouth, N.H.) is dissolved in aqueous acetic acid by stirring with a mechanical stirrer for one day. Nitrogen gas is bubbled through the solution, while an aqueous solution of sodium nitrite is added. After a half hour, the solution is filtered through a sintered glass funnel, under reduced pressure, to remove insoluble particles which are present in the initial chitosan solution. To the filtered solution is added an aqueous solution of NaOH, and the solution is vigorously stirred in methanol to precipitate the polymer. The resulting precipitate is then filtered and alternately washed five times with water and methanol. The precipitate is then dried in a vacuum oven at 60° C. for two days. The depolymerized chitosan comprises an aldehyde group at one end of the chain. The aldehyde end group may be reduced to a primary hydroxyl group by reaction NaBH₄. The depolymerized product can be analyzed by gel permeation chromatography (GPC) to determine both its molecular weight and molecular weight distribution (MWD) in comparison to Pullulan reference standards. NMR (nuclear magnetic resonance) and IR (infra-red) studies can be used to determine the amount of N-acetylation on the depolymerized product.

(ii) Partial Succinylation of Depolymerized Chitosan

The depolymerized chitosan from (i) is dissolved in 0.1M aqueous acetic acid. To this solution, methanol is added followed by the addition of a solution of succinic anhydride in acetone. The resulting solution is stirred at room temperature for 24 hours. Upon completion of the succinylation, the solution is then precipitated into aqueous acetone. The resulting precipitate is collected by centrifugation and washed five times with methanol. The precipitate is then dissolved in 0.5M KOH and dialyzed against water to a pH of 7. The dialyzed solution is then concentrated under reduced pressure, precipitated in aqueous acetone, and dried in a vacuum oven at 60° C.

To obtain variable levels of succinylation, the extent of the reaction can be monitored as the acylation proceeds by analyzing for number of unacylated amine groups. The number of unacylated amine groups can be determined by quenching a withdrawn sample of the reaction mixture with an amine detecting agent (e.g., flouorescamine). The amount of amine present can be measured spectrophoretically using a standard curve for the copolymer. Succinic anhydride can thus be added successively until the desired acylation percentage is achieved. The exact degree of succinylation of the purified product can be determined using sup.¹H NMR spectroscopy and conductometric titration.

(iii) Conjugation of VRA 1 to Succinylated Chitosan

The above succinylated chitosan (100 mg) is dissolved in 2 ml water, to which 10 mg sodium salt of VRA 1 dissolved in 1 ml DMF is added. To this solution is added 1-(3-dimethylaminopropyl)-3-ethyl-carbodiimide. The reaction mixture is left stirring for 24 h and the resulting solution is first dialyzed against (1K molecular weight cut off) 2L of Bup MES (Pierce) solution having a pH of 4.7, and then dialyzed against milliQ water. The dialyzate is lyophilized to obtain the carboxymethyl chitosan VRA 1 conjugate.

All publications, including but not limited to patents and patent applications cited in this specification are herein incorporated by reference as if each individual publication were specifically and individually indicated to be incorporated by reference as though fully set forth.

1. A polymer-therapeutic comprising:
(a) a polymer-receptor antagonist conjugate comprising:
(i) a polymeric component selected from the group consisting of hydrophilic polymers, hydrophobic polymers, and amphiphilic copolymers, and
(ii) a non-biological, biomimetic antagonist to a receptor upregulated at a disease site, chemically linked to the polymeric component; and
(b) a pharmaceutical active.

2. A polymer therapeutic according to claim 1 wherein the polymeric component is a hydrophilic polymer.

3. A polymer therapeutic according to claim 2 wherein the hydrophilic polymer is selected from the group consisting of polyalkyl ethers; alkoxy—capped polyalkyl ethers; polyvinylpyrrolidones; polyvinylalkyl ethers; polyoxazolines; polyalkyl oxazolines; polyhydroxyalkyl oxazolines; polyacrylamides; polyalkyl acrylamides; polyhydroxyalkyl acrylamides; polyhydroxyalkyl acrylates; polysialic acids and analogs thereof; hydrophilic peptide sequences; polysaccharides; polyaminoacids thereof; maleic anhydride copolymers; polyvinylalcohols; copolymers of any of the foregoing polymers; and derivatives of any of the foregoing polymers and copolymers.

4. A polymer therapeutic according to claim 1 wherein the polymeric component is an amphiphilic copolymer.

5. A polymer therapeutic according to claim 4 wherein the amphiphilic copolymer comprises:
(a) a hydrophilic polymer segment selected from the group consisting of polyalkyl ethers; alkoxy—capped polyalkyl ethers; polyvinylpyrrolidones; polyvinylalkyl ethers; polyoxazolines; polyalkyl oxazolines; polyhydroxyalkyl oxazolines; polyacrylamides; polyalkyl acrylamides; polyhydroxyalkyl acrylamides; polyhydroxyalkyl acrylates; polysialic acids and analogs thereof; hydrophilic peptide sequences; polysaccharides; polyaminoacids; maleic anhydride copolymers; polyvinylalcohols; copolymers of any of the foregoing polymers; and derivatives of any of the foregoing polymers and copolymers; and
(b) a hydrophobic polymer segment selected from the group consisting of polyesters, polycarbonates, polyanhydrides, polyorthoesters, polypropylene glycol, hydrophobic derivatives of poly(alpha-amino acids), copolymers of any of the foregoing polymers, and derivatives of any of the foregoing polymers and copolymers.

6. A polymer therapeutic according to claim 5 wherein the amphiphilic copolymer comprises:
(a) a hydrophilic polymer segment selected from the group consisting of polyethylene glycol, polyvinylpyrrolidone, polyacrylamide, poly(hydroxypropyl acrylamide), polyvinylalcohol, polysaccharides, polyaminoacids, polyoxazolines, copolymers of any of the foregoing polymers, and derivatives of any of the foregoing polymers and copolymers; and
(b) a hydrophobic polymer segment selected from the group consisting of polyesters, polycarbonates, polyanhydrides, polyorthoesters, polypropylene glycol, hydrophobic derivatives of poly(alpha-amino acids), copolymers of any of the foregoing polymers, and derivatives of any of the foregoing polymers and copolymers.

7. A polymer therapeutic according to claim 6 wherein the amphiphilic copolymer comprises a hydrophilic polyethylene glycol segment and a hydrophobic polyester segment.

8. A polymer therapeutic according to claim 1 wherein the non-biological, biomimetic antagonist is an antagonist to a receptor upregulated in the vascular endothelium of inflammation, infection or tumor sites.

9. A polymer therapeutic according to claim 1 wherein the non-biological, biomimetic antagonist is an antagonist to a receptor selected from the group consisting of αVβ3, αVβ5, α5β1, Prostate Specific Membrane Antigen (PSMA) receptor, Herceptin, Tie1 receptor, Tie2 receptor, ICAM1, Folate receptor, basic Fibroblast Growth Factor (bFGF) receptor, Epidermal Growth Factor (EGF) receptor, Vascular Endothelial Growth Factor (VEGF), Platelet Derived Growth Factor (PDGF) receptor, Laminin receptor, Endoglin, Vascular Cell Adhesion Molecule VCAM-1, E-Selectin, and P-Selectin.

10. A polymer therapeutic according to claim 9 wherein the non-biological, biomimetic antagonist is an antagonist to a receptor selected from the group consisting of αVβ3, αVβ5 and α5β1.

11. A polymer therapeutic according to claim 1 wherein the non-biological, biomimetic antagonist is selected from the group consisting of analogs of YIGSR-NH2, PD 156707 and derivatives thereof, and integrin receptor antagonists.

12. A polymer therapeutic according to claim 1 wherein the non-biological, biomimetic antagonist is a vitronectin receptor antagonist.

13. A polymer therapeutic according to claim 12 wherein the receptor antagonist is:

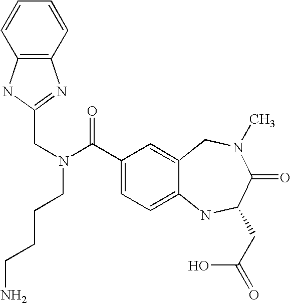

14. A polymer-therapeutic according to claim 1, wherein the polymeric-therapeutic comprises a polymeric micelle comprising said polymer-receptor antagonist conjugate, and the polymer-receptor antagonist conjugate comprises:
(a) an amphiphilic copolymer having a hydrophilic terminus, and
(b) a non-biological, biomimetic antagonist to a receptor upregulated at a disease site, chemically linked to the copolymer hydrophilic terminus.

15. A polymer-receptor antagonist conjugate useful for preparing polymer-therapeutics, comprising:
(a) a polymer selected from the group consisting of hydrophilic polymers, hydrophobic polymers, and amphiphilic copolymers; chemically linked to
(b) a non-biological, biomimetic antagonist to a receptor upregulated at a disease site.

16. A polymeric micelle comprising a polymer-receptor antagonist conjugate according to claim 15.

17. A method of treating or diagnosing a disease characterized by upregulation of a receptor, comprising administering to a patient in need thereof a safe and effective amount of a polymer-therapeutic according to claim 1, wherein the antagonist has binding affinity to the upregulated receptor.

18. A method according to claim 17 wherein the receptor is upregulated in the vascular endothelium of inflammation, infection or tumor sites.

19. A method according to claim 18 wherein the receptor is an integrin.

20. A method according to claim 19 wherein the receptor is the vitronectin receptor.

21. A method according to claim 17 wherein the disease is characterized by angiogenesis.

22. A process for preparing an amphiphilic biodegradable polymer having carboxylic groups at the hydrophilic terminus, comprising reacting a hydrophilic, alpha hydroxy omega carboxylic polyalkyleneglycol and a hydrophobic cyclic monomer such that ring opening polymerization of the monomer is initiated by the polyalkylene glycol hydroxy terminus.

23. A process according to claim 22 wherein the hydrophobic cyclic monomer is selected from the group consisting of propylene oxide, lactide, caprolactone, dioxanone, trimethylene carbonate, and combinations thereof.

24. A process according to claim 22 wherein the polyalkyleneglycol is polyethylene glycol.

25. A process according to claim 22 wherein the reaction occurs in the presence of a catalyst and at a temperature of from about 100° C. to about 200° C.

26. A process according to claim 25 wherein the catalyst is a transition metal catalyst.
